(19) United States
(12) Patent Application Publication
(10) Pub. No.: US 2023/0045126 A1
(43) Pub. Date: Feb. 9, 2023

(54) DEVICES AND METHODS FOR DETERMINING NUCLEIC ACIDS USING DIGITAL DROPLET PCR AND RELATED TECHNIQUES

(71) Applicant: President and Fellows of Harvard College, Cambridge, MA (US)

(72) Inventors: David A. Weitz, Cambridge, MA (US); Huidan Zhang, Cambridge, MA (US)

(73) Assignee: President and Fellows of Harvard College, Cambridge, MA (US)

(21) Appl. No.: 17/790,450

(22) PCT Filed: Jan. 13, 2021
(86) PCT No.: PCT/US2021/013231

§ 371 Date: Jun. 30, 2022

Related U.S. Application Data
(60) Provisional application No. 62/961,097, filed on Jan. 14, 2020.

(51) Int. Cl. C12Q 1/6806; C12Q 1/6876; C12Q 1/686; B01L 3/00; C12Q 1/6834

(52) U.S. Cl. CPC C12Q 1/6806; C12Q 1/6876; C12Q 1/686; B01L 3/5027; C12Q 1/6834

(57) ABSTRACT

The present disclosure generally relates, in certain aspects, to droplet-based microfluidic devices and methods. In certain aspects, target nucleic acids contained within droplets are amplified within droplets in a first step, where multiple primers may be present. However, multiple primers may cause multiple target nucleic acids to be amplified within the droplets, which can make it difficult to identify which nucleic acids were amplified. In a second step, the amplified nucleic acids may be determined. For example, the droplets may be broken and the amplified nucleic acids can be pooled together and sequenced. As an example, new droplets may be formed containing the amplified nucleic acids, and those nucleic acids within the droplets amplified by exposure to certain primers.

### RELATED APPLICATIONS

This application claims the benefit of U.S. Provisional Patent Application Ser. No. 62/961,097, filed Jan. 14, 2020, entitled “Devices and Methods for Determining Nucleic Acids Using Digital Droplet PCR and Related Techniques,” by Weitz, et al., incorporated herein by reference in its entirety.

### GOVERNMENT FUNDING

This invention was made with government support under Grant No. 1420570 awarded by the National Science Foundation. The government has certain rights in the invention.

### FIELD

The present disclosure generally relates, in certain aspects, to droplet-based microfluidic devices and methods. In some cases, digital amplification through PCR is used.

### BACKGROUND

A variety of techniques exist for producing fluidic droplets within a microfluidic system, such as those disclosed in Int. Pat. Pub. Nos. WO 2004/091763, WO 2004/002627, WO 2006/096571, WO 2005/021151, WO 2010/033200, and WO 2011/056546, each incorporated herein by reference in its entirety. In some cases, relatively large numbers of droplets may be produced, and often at relatively high speeds, e.g., the droplets may be produced at rates of least about 10 droplets per second. The droplets may also contain a variety of species therein. However, improvements in determining the species within the droplets are needed.

### SUMMARY

The present disclosure generally relates, in certain aspects, to droplet-based microfluidic devices and methods. In some cases, digital amplification through PCR is used. The subject matter of the present disclosure involves, in some cases, interrelated products, alternative solutions to a particular problem, and/or a plurality of different uses of one or more systems and/or articles.

Some aspects are generally directed to certain methods. For example, in one embodiment, the method comprises forming a first plurality of droplets, at least 90% of which contain either only one target nucleic acid or no target nucleic acid, and at least 90% of which contain at least one amplification primer; amplifying the target nucleic acids within the first plurality of droplets using the at least one amplification primer to produce amplified nucleic acids; breaking the first plurality of droplets to mix the amplified nucleic acids; forming a second plurality of droplets, at least 90% of which contains either one of the amplified nucleic acids or no amplified nucleic acid, and at least 90% of which contain at least one selection primer; amplifying the amplified nucleic acids within the second plurality of droplets using the at least one selection primer to produce determinable nucleic acids; and determining at least some of the determinable nucleic acids.

In another embodiment, the method comprises forming a plurality of droplets, at least 90% of which contain either only one target nucleic acid or no target nucleic acid, and at least 90% of which contain a plurality of different amplification primers; amplifying the target nucleic acids within the plurality of droplets using the plurality of amplification primers to produce amplified nucleic acids; breaking the droplets to form a mixture of the amplified nucleic acids; and determining at least some of the amplified nucleic acids within the mixture.

In another aspect, the present disclosure encompasses methods of making one or more of the embodiments described herein, for example, for digital droplet PCR and other applications. In still another aspect, the present disclosure encompasses methods of using one or more of the embodiments described herein, for example, for digital droplet PCR and other applications.

Other advantages and novel features will become apparent from the following detailed description of various non-limiting embodiments when considered in conjunction with the accompanying figures.

### BRIEF DESCRIPTION OF THE DRAWINGS

Non-limiting embodiments will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the embodiment. In the figures:

FIGS. 1A-1E illustrates single-molecule characterization of each individual mutant amplicon using barcoded droplets, in accordance with one embodiment;

FIGS. 2A-2B illustrate Sanger sequencing, in another embodiment;

FIGS. 3A-3B illustrate next generation sequencing, in yet another embodiment; and

FIG. 4 illustrates hybridization, in still another embodiment.

### DETAILED DESCRIPTION

The present disclosure generally relates, in certain aspects, to droplet-based microfluidic devices and methods. In certain aspects, target nucleic acids contained within droplets are amplified within droplets in a first step, where multiple primers may be present. However, multiple primers may cause multiple target nucleic acids to be amplified within the droplets, which can make it difficult to identify which nucleic acids were amplified. In a second step, the amplified nucleic acids may be determined. For example, the droplets may be broken and the amplified nucleic acids can be pooled together and sequenced. As an example, new droplets may be formed containing the amplified nucleic acids, and those nucleic acids within the droplets amplified by exposure to certain primers. This may be useful, for example to determine whether a certain target nucleic acid is present within a sample, e.g., even if the target nucleic acid is present in very low concentrations. In addition, in some cases, the droplets may be divided into different groups, such that the groups are exposed to different primers. Still other sequencing techniques can be used in other embodiments. The second step may allow for much larger multiplexing, to increase the specificity and/or selectivity of the amplified nucleic acids, etc.

Some aspects are generally directed to systems and methods of determining target nucleic acids in a sample. In some cases, the targets may be present at very low concentrations. For instance, a target nucleic acid may be present in a sample containing other nucleic acids at a concentration of 1:10³, 1:10⁴, 1:10⁵, 1:10⁶, 1:10⁷, 1:10⁸, or even lower concentrations.

In some cases, the nucleic acids may be amplified in some fashion. For instance, the nucleic acids may be encapsulated into droplets. In some cases, the nucleic acids are encapsulated at relatively low concentrations, e.g., such that the droplets may, on the average contain less 1 nucleic acid per droplet. This may be useful to ensure that most or all of the nucleic acids are amplified, e.g., substantially evenly. In contrast, if the nucleic acids were to be amplified in bulk solution, some nucleic acids could be amplified without others being amplified (or being amplified to a much lesser degree). Thus, in certain embodiments as described herein the nucleic acids are encapsulated into droplets, and amplified therein.

In some cases, a plurality of primers may be added to the droplets to cause amplification, e.g., using droplet-based PCR or other techniques known to those of ordinary skill in the art. In some cases, there may be at least 3, at least 5, at least 10, at least 30, at least 50, at least 100, at least 300, at least 500, at least 1,000, at least 2,000, at least 3,000, at least 5,000, or at least 10,000, or more distinguishable primers present. This may be useful, for example, to ensure a large number of potential target nucleic acids are amplified. However, it can make it difficult to identify which nucleic acids were amplified.

Accordingly, in a second step, the amplified droplets may be determined or sequenced, e.g., using any of a variety of techniques. For instance, in one set of embodiments, the droplets may be broken and their contents pooled together, e.g., to create a pool of amplified nucleic acids. The pool of amplified nucleic acids may then be sequenced or determined (e.g., qualitatively or quantitatively), for example, using techniques such as Sanger sequencing, Illumina sequencing, DNA microarrays, single-molecule real-time sequencing (e.g., Pacbio sequencing), nanopore sequencing, capillary electrophoresis, or the like. Determination of nucleic acids may include, as non-limiting examples, determining whether nucleic acid or a class of nucleic acids is present, determining some or all of the sequence of the nucleic acid, determining a concentration of the nucleic acid, etc. In some cases, the pool of amplified nucleic acids may be determined or identified, e.g., without any sequencing.

In addition, in certain embodiments, the pool of amplified nucleic acids may be sequenced using droplet-based techniques, e.g., droplet-based PCR. For example, in some cases, the amplified nucleic acids may be collected into droplets and the droplets exposed to certain primers, e.g., primers that are able to amplify rare target nucleic acid sequences. In some cases, the amplified nucleic acids may be collected into droplets at relatively low concentrations, e.g., such that the droplets may, on the average, contain less than 1 nucleic acid per droplet or less than 1 target per droplet, for instance, as described herein. In addition, in certain embodiments, the droplets may be divided into different groups of droplets, which are exposed to different primers. For instance, the droplets may be divided into at least 5, 10, 30, 100, etc. groups, which are exposed to various primers, e.g., in different spatial locations, to determine whether a target nucleic acid was present in the sample. However, it should be understood that in other embodiments, the amplified nucleic acids may be present at relatively higher concentrations, e.g., at at least 1 nucleic acid per droplet or at at least 1 target per droplet. In some cases, more than one primer or one amplicon may be present within a droplet.

In one aspect, for example, a sample containing oligonucleotides, or other nucleic acids (including those described below), is encapsulated into droplets. These may, for example, be targets that are to be determined within the sample, e.g., qualitatively and/or quantitatively. The oligonucleotides are amplified within the droplets, e.g., using PCR or other techniques. For example, a large number of primers may be present or added to at least some of the droplets, e.g., which may allow for relatively large variety of oligonucleotides to be amplified within each droplet. In some cases, the oligonucleotides are distributed within the droplets at a very low density, e.g., such that most or all of the droplets contain only a single oligonucleotide or no oligonucleotide. Such a system may be useful, for example, to produce a larger number or concentration of oligonucleotides for subsequent analysis, e.g., as discussed below. Using a relatively large number of primers may allow for the amplification of a large range of possible oligonucleotides, while isolating individual oligonucleotides within separate droplets may allow for the amplification of oligonucleotides in a relatively even manner, e.g., such that most or all of the oligonucleotides will be amplified, for instance, without competitive effects that may occur when two or more oligonucleotides are being amplified together.

After amplification, the droplets may be broken and their contents combined together, thereby producing a mixture of amplified oligonucleotides. The oligonucleotides may then be determined in some manner. A variety of techniques may be used to determine the oligonucleotides, quantitatively and/or qualitatively, such as Sanger sequencing, Illumina sequencing, DNA microarrays, single-molecule real-time sequencing (e.g., Pacbio sequencing), nanopore sequencing, capillary electrophoresis, or the like.

As another non-limiting example, a second stage of amplification within droplets may be performed, e.g., to facilitate determination and/or sequencing of the oligonucleotides. The mixture of amplified oligonucleotides, in accordance with certain embodiments, may again be contained within droplets, and then amplified within the droplets. In some cases, the amplified oligonucleotides may be contained within the droplets at a relatively low density, e.g., such that most or all of the droplets contain only a single oligonucleotide or no oligonucleotide. In some embodiments, the amplification within the droplets may also be relatively selective, e.g., by using one or more primers that only allow certain types of oligonucleotides to be amplified. Thus, for example, primers that allow only mutants of a certain oligonucleotide sequence may be present at this stage, and thus, oligonucleotides having sufficient similarity to the sequence may be amplified using the primers, while other oligonucleotides, such as contaminants or irrelevant sequences, may not be amplified within the droplets. After amplification, the amplified oligonucleotides may optionally be sequenced, e.g., using techniques such as those described herein, or otherwise analyzed. In some cases, the droplets may be divided into different groups, at least some of which may be exposed to different primers, e.g., to determine whether different types of target oligonucleotides are present within a sample.

The above discussion illustrates non-limiting examples certain embodiments that can be used to determine or sequence oligonucleotides from a sample. However, other embodiments are also possible. Accordingly, more generally, some aspects are directed to various systems and methods for determining or sequencing nucleic acids, such as oligonucleotides, from a sample.

A variety of target nucleic acids may be determined in accordance with various aspects, including oligonucleotides. The nucleic acids may arise from a cell, such as a mammalian cell, or from other sources. The nucleic acids may be, for example, RNA and/or DNA, such as genomic DNA or mitochondrial DNA. In some cases, the nucleic acids are free-floating or contained within a fluid contained within the droplet. The nucleic acid may be taken from one or more cells (e.g., released upon lysis of one or more cells), synthetically produced, or the like. If the nucleic acid arises from cells, the cells may come from the same or different species (e.g., mouse, human, bacterial, etc.), and/or the same or different individual. For example, the nucleic acids may come from cells of a single organism, e.g., healthy or diseased cells (e.g., cancer cells), different organs of the organism, etc. In some cases, different organisms may be used (e.g., of the same or different species). In some cases, the nucleic acids may have a distribution such that some nucleic acids are not commonly present within a nucleic acid population. For example, there may be one cancer or disease cell among tens, hundreds, thousands, or more of normal or other cells.

For instance, in one set of embodiments, one or more cells may be lysed, and nucleic acids from the cells may be collected and distributed or encapsulated into droplets, e.g., as discussed herein. The lysing can be performed using any suitable technique for lysing cells. Non-limiting examples include ultrasound or exposure to suitable agents such as surfactants. In some cases, the exact technique chosen may depend on the type of cell being lysed; many such cell lysing techniques will be known by those of ordinary skill in the art.

The cells may arise from any suitable source. For instance, the cells may be any cells for which nucleic acid from the cells is desired to be studied or sequenced, etc., and may include one, or more than one, cell type. The cells may be for example, from a specific population of cells, such as from a certain organ or tissue (e.g., cardiac cells, immune cells, muscle cells, cancer cells, etc.), cells from a specific individual or species (e.g., human cells, mouse cells, bacteria, etc.), cells from different organisms, cells from a naturally-occurring sample (e.g., pond water, soil, etc.), or the like. In some cases, the cells may be dissociated from tissue.

In one set of embodiments, a sample containing nucleic acids may be contained within a plurality of droplets, e.g., contained within a suitable carrying fluid. The nucleic acids may be present during formation of the droplets, and/or added to the droplets after formation. Any suitable method may be chosen to create droplets, and a wide variety of different droplet makers and techniques for forming droplets will be known to those of ordinary skill in the art. For example, a junction of channels may be used to create the droplets. The junction may be, for instance, a T-junction, a Y-junction, a channel-within-a-channel junction (e.g., in a coaxial arrangement, or comprising an inner channel and an outer channel surrounding at least a portion of the inner channel), a cross (or “X”) junction, a flow-focusing junction, or any other suitable junction for creating droplets. See, for example, International Patent Application No. PCT/US2004/010903, filed Apr. 9, 2004, entitled “Formation and Control of Fluidic Species,” by Link, et al., published as WO 2004/091763 on Oct. 28, 2004, or International Patent Application No. PCT/US2003/020542, filed Jun. 30, 2003, entitled “Method and Apparatus for Fluid Dispersion,” by Stone, et al., published as WO 2004/002627 on Jan. 8, 2004, each of which is incorporated herein by reference in its entirety.

In certain embodiments, nucleic acids may be added to droplet after the droplet has been formed, e.g., through picoinjection or other methods such as those discussed in Int. Pat. Apl. Pub. No. WO 2010/151776, entitled “Fluid Injection” (incorporated herein by reference), through fusion of the droplets with droplets containing the nucleic acids, or through other techniques known to those of ordinary skill in the art.

The nucleic acids may be contained within the droplets at relatively low densities, in accordance with certain embodiments. For instance, in one set of embodiments, the droplets may, on the average contain less 1 nucleic acid per droplet. For example, the average loading rate may be less than about 1 particle/droplet, less than about 0.9 nucleic acids/droplet, less than about 0.8 nucleic acids/droplet, less than about 0.7 nucleic acids/droplet, less than about 0.6 nucleic acids/droplet, less than about 0.5 nucleic acids/droplet, less than about 0.4 nucleic acids/droplet, less than about 0.3 nucleic acids/droplet, less than about 0.2 nucleic acids/droplet, less than about 0.1 nucleic acids/droplet, less than about 0.05 nucleic acids/droplet, less than about 0.03 nucleic acids/droplet, less than about 0.02 nucleic acids/droplet, or less than about 0.01 nucleic acids/droplet. In some cases, lower densities may be chosen to minimize the probability that a droplet will have two or more nucleic acids in it. Thus, for example, at least about 50%, at least about 60%, at least about 70%, at least about 80%, at least about 90%, at least about 95%, at least about 97%, at least about 98%, or at least about 99% of the droplets may contain either no target nucleic acid or only one such nucleic acid.

However, in some cases, the loading densities may also be controlled such that at least a signification amount of the droplets contains a target nucleic acid. This may be useful, for example, to prevent too much inefficiency in loading, or subsequent operations, etc. For instance, at least about 50%, at least about 60%, at least about 70%, at least about 80%, or at least about 90% of the droplets may also contain at least one such nucleic acid.

In some cases, the nucleic acids within the droplets may be amplified. This may be useful, for example, to produce a larger number or concentration of nucleic acids, e.g., for subsequent analysis, sequencing, or the like. Those of ordinary skill in the art will be familiar with various amplification methods that can be used, including, but are not limited to, polymerase chain reaction (PCR), reverse transcriptase (RT) PCR amplification, in vitro transcription amplification (IVT), multiple displacement amplification (MDA), or quantitative real-time PCR (qPCR).

In some cases, the nucleic acids may be amplified within the droplets. This may allow amplification to occur “evenly” in some embodiments, e.g., such that the distribution of nucleic acids is not substantially changed after amplification, relative to before amplification. For example, according to certain embodiments, the nucleic acids within a plurality of droplets may be amplified such that the number of nucleic acid molecules for each type of nucleic acid may have a distribution such that, after amplification, no more than about 5%, no more than about 2%, or no more than about 1% of the nucleic acids have a number less than about 90% (or less than about 95%, or less than about 99%) and/or greater than about 110% (or greater than about 105%, or greater than about 101%) of the overall average number of amplified nucleic acid molecules per droplet. In some embodiments, the nucleic acids within the droplets may be amplified such that each of the nucleic acids that are amplified can be detected in the amplified nucleic acids, and in some cases, such that the mass ratio of the nucleic acid to the overall nucleic acid population changes by less than about 50%, less than about 25%, less than about 20%, less than about 15%, less than about 10%, or less than about 5% after amplification, relative to the mass ratio before amplification.

In some cases, certain primers are contained within the droplets to promote amplification. Such primers may be present during formation of the droplets, and/or added to the droplets after formation of the droplets. It should be noted that the manner in which the primers are added to the droplets may be the same or different from the manner in which the nucleic acids are added to the droplets.

In certain embodiments, a plurality of different types of primers may be added to the droplets. For instance, the primers may be distinguishable due to their having different sequences, and/or such that they are able to amplify different potential targets. In some cases, at least 2, at least 3, at least 5, at least 10, at least 15, at least 20, at least 25, at least 30, at least 40, at least 50, at least 60, at least 75, at least 100, at least 150, at least 200, at least 300, at least 400, at least 500, at least 1,000, at least 2,000, at least 3,000, at least 5,000, or at least 10,000, etc., different primers may be used. This may allow, for example, a variety of different target nucleic acids to be amplified within different droplets.

Examples of techniques for forming droplets include those described above. Examples of techniques for introducing primers after droplet formation include picoinjection or other methods such as those discussed in Int. Pat. Apl. Pub. No. WO 2010/151776, incorporated herein by reference, through fusion of the droplets with droplets containing primers, or the like. Other such techniques for either of these include, but are not limited to, any of those techniques described herein.

The primers may be present within the droplets at any suitable density. The density may be independent of the density of target nucleic acids. In some cases, an excess of primers are used, e.g., such that the target nucleic acids controls the reaction. For instance, if a large excess of primers are used, then substantially of the droplets will contain primer (regardless of whether or not the droplets also contain target nucleic acids). For example, in certain embodiments, at least about 50%, at least about 60%, at least about 70%, at least about 80%, at least about 90%, at least about 95%, at least about 97%, at least about 98%, or at least about 99% of the droplets may contain at least one amplification primer.

Droplets containing both primer and a target nucleic acid may be treated to cause amplification of the nucleic acid to occur. This may allow a large amount or concentration of the target nucleic acids to be produced, e.g., without substantially altering the distribution of nucleic acids. In some cases, the primers are selected to allow substantially all, or only some, of the target nucleic acids suspected of being present to be amplified.

As examples, PCR (polymerase chain reaction) or other amplification techniques may be used to amplify nucleic acids, e.g., contained within droplets. Typically, in PCR reactions, the nucleic acids are heated (e.g., to a temperature of at least about 50° C., at least about 70° C., or least about 90° C. in some cases) to cause dissociation of the nucleic acids into single strands, and a heat-stable DNA polymerase (such as Taq polymerase) is used to amplify the nucleic acid. This process is often repeated multiple times to amplify the nucleic acids.

Thus, in one set of embodiments, PCR amplification may be performed within the droplets. For example, the droplets may contain a polymerase (such as Taq polymerase), and DNA nucleotides (deoxyribonucleotides), and the droplets may be processed (e.g., via repeated heated and cooling) to amplify the nucleic acid within the droplets. Suitable reagents for PCR or other amplification techniques, such as polymerases and/or deoxyribonucleotides, may be added to the droplets during their formation, and/or afterwards (e.g., via merger with droplets containing such reagents, and/or via direct injection of such reagents, e.g., contained within a fluid). Various techniques for droplet injection or merger of droplets will be known to those of ordinary skill in the art. See, e.g., U.S. Pat. Apl. Pub. No. 2012/0132288, incorporated herein by reference. In some embodiments, primers may be added to the droplets, or the primers may be present on one or more of the nucleic acids within the droplets. Those of ordinary skill in the art will be aware of suitable primers, many of which can be readily obtained commercially.

In one set of embodiments, at least some of the primers may be distinguished, for example, using distinguishable fluorescent tags, barcodes, or other suitable identification tags. Examples of barcodes that can be contained within droplets include, but are not limited to, those described in U.S. Pat. Apl. Pub. No. 2018-0304222 or Int. Pat. Apl. Pub. No. WO 2015/164212, each incorporated herein by reference.

The nucleic acids may be amplified to any suitable extent. The degree of amplification may be controlled, for example, by controlling factors such as the temperature, cycle time, or amount of enzyme and/or deoxyribonucleotides contained within the droplets. For instance, in some embodiments, a population of droplets may have at least about 50,000, at least about 100,000, at least about 150,000, at least about 200,000, at least about 250,000, at least about 300,000, at least about 400,000, at least about 500,000, at least about 750,000, at least about 1,000,000 or more molecules of the amplified nucleic acid per droplet.

In one set of embodiments, the droplets are broken down after amplification, e.g., to allow the amplified nucleic acids to be pooled together. A wide variety of methods for “breaking” or “bursting” droplets are available to those of ordinary skill in the art. For example, droplets contained in a carrying fluid may be disrupted using techniques such as mechanical disruption, chemical disruption, or ultrasound. Droplets may also be disrupted using chemical agents or surfactants, for example, 1H,1H,2H,2H-perfluorooctanol.

After amplification, one or more of the nucleic acids may be determined or sequenced. However, it should be noted that because there are larger numbers of nucleic acids present, e.g., due to amplification, such analysis can be much easier. Such analysis can take many different forms in various embodiments, for instance, depending on factors such as the nature of the detection, the degree of quantification required, or the like.

Examples of methods for determining and/or sequencing nucleic acids include, but are not limited to, chain-termination sequencing, sequencing-by-hybridization, Maxam-Gilbert sequencing, dye-terminator sequencing, chain-termination methods, Massively Parallel Signature Sequencing (Lynx Therapeutics), polony sequencing, pyrosequencing, sequencing by ligation, ion semiconductor sequencing, DNA nanoball sequencing, single-molecule real-time sequencing (e.g., Pacbio sequencing), nanopore sequencing, Sanger sequencing, digital RNA sequencing (“digital RNA-seq”), Illumina sequencing, capillary electrophoresis, etc. In some cases, a microarray, such as a DNA microarray, may be used, for example to determine or identify nucleic acids. Those of ordinary skill in the art will be aware of other techniques that can be used to determine and/or sequence nucleic acids, e.g., qualitatively and/or quantitatively.

In addition, in some cases, the nucleic acids may be determined using droplet-based techniques, e.g., droplet-based PCR. As an example, the amplified nucleic acids may be contained within droplets, in accordance with certain embodiments, e.g., for subsequent analysis. The droplets may be created using any suitable technique, such as those described herein, and the technique for creating these droplets may be the same or different than for the initial droplets. In some cases, the droplets may also be monodisperse, and/or have distributions or dimensions such as are described herein. The amplified nucleic acids may be contained within droplets using any suitable technique, e.g., during or after the droplets have been formed. Techniques for creating droplets and/or adding fluid to a droplet have been discussed herein.

In some cases, the amplified nucleic acids may be contained within droplets at relatively low densities. For example, the droplets may, on the average contain less 1 nucleic acid per droplet. For example, the average loading rate may be less than about 1 particle/droplet, less than about 0.9 nucleic acids/droplet, less than about 0.8 nucleic acids/droplet, less than about 0.7 nucleic acids/droplet, less than about 0.6 nucleic acids/droplet, less than about 0.5 nucleic acids/droplet, less than about 0.4 nucleic acids/droplet, less than about 0.3 nucleic acids/droplet, less than about 0.2 nucleic acids/droplet, less than about 0.1 nucleic acids/droplet, less than about 0.05 nucleic acids/droplet, less than about 0.03 nucleic acids/droplet, less than about 0.02 nucleic acids/droplet, or less than about 0.01 nucleic acids/droplet. In some cases, lower densities may be chosen to minimize the probability that a droplet will have two or more nucleic acids in it. Thus, for example, at least about 50%, at least about 60%, at least about 70%, at least about 80%, at least about 90%, at least about 95%, at least about 97%, at least about 98%, or at least about 99% of the droplets may contain either no target nucleic acid or only one such nucleic acid. In addition, in some cases, the loading densities may also be controlled such that at least a signification amount of the droplets contains a target nucleic acid. This may be useful, for example, to prevent too much inefficiency in loading, or subsequent operations, etc. For instance, at least about 50%, at least about 60%, at least about 70%, at least about 80%, or at least about 90% of the droplets may also contain at least one such nucleic acid.

In some embodiments, a second stage of amplification within droplets may be performed. The amplification within the droplets may also be relatively selective, for example, for quantitative detection, or for the determination of specific sequences, for example, by providing only certain primers. For instance, one or only a relatively small number of primers (e.g., no more than 20, 15, 10, 5, 3, or 2) may be provided in certain embodiments, thereby allowing only specific nucleic acid sequences to be amplified, e.g., within the droplets. In some cases, at least 3, 4, 5, 10, 15, or 20 primers may be present.

As a non-limiting example, primers that allow only certain mutations in a nucleic acid to be amplified may be used during amplification. For instance, a plurality of primers may be used that have relatively small differences, e.g. such that the primers have at least 70%, at least 75%, at least 80%, at least 85%, at least 90%, or at least 95% homology, and/or such that the amplification primers are all substantially identical except for no more than 5, 4, 3, 2, or 1 nucleotide differences. In addition, in certain cases, a blocking nucleotide that prevents amplification of the non-mutated nucleic acid may also be used, e.g., to allow only the mutated nucleic acid to be amplified.

In some cases, the primers, if used, may be contained within the droplets using techniques such as those described herein. For instance, the primers may be present during formation of the droplets, and/or added to the droplets after formation of the droplets. It should be noted that the manner in which the primers are added to the droplets may be the same or different from the manner in which the nucleic acids are added to the droplets, and/or from the manner in which primers were added to the initial droplets.

In certain embodiments, the primers may be distributed such that some or all of the droplets contains only a single primer. For instance, at least about 50%, at least about 60%, at least about 70%, at least about 80%, at least about 90%, at least about 95%, at least about 97%, at least about 98%, or at least about 99% of the droplets may contain either no primer or only a single primer. In some cases, at least about 50%, at least about 60%, at least about 70%, at least about 80%, or at least about 90% of the droplets may contain only a single primer.

In one set of embodiments, at least some of the primers may be distinguished, for example, using distinguishable fluorescent tags, barcodes, or other suitable identification tags. Examples of barcodes that can be contained within droplets include, but are not limited to, those described in U.S. Pat. Apl. Pub. No. 2018-0304222 or Int. Pat. Apl. Pub. No. WO 2015/164212, each incorporated herein by reference.

In some embodiments, a plurality of different droplet makers may be used, each of which introduces a single primer into the droplets as they are formed. Examples of droplet makers include channel junctions such as a T-junction, a Y-junction, a channel-within-a-channel junction, a cross (or “X”) junction, a flow-focusing junction, or the like. Other suitable examples of different droplet makers and techniques for forming droplets include any of those discussed herein. Examples of techniques for introducing primers after droplet formation include picoinjection or other methods such as those discussed in Int. Pat. Apl. Pub. No. WO 2010/151776, incorporated herein by reference, through fusion of the droplets with droplets containing primers, or the like.

In some cases, the droplets may be divided into different groups such that the droplets are exposed to different primers, e.g., that are injected into the droplets. However, in other embodiments, the primers may be distributed differently, e.g., such that some or all of the droplets contains some or all of the primers.

Thus, in some embodiments, even though the primers may be distributed such that some or all of the droplets contains only a single primer in certain embodiments, because different groups of droplets are used, a plurality of different targets may still be determined for a pool of amplified nucleic acids. For instance, the droplets may be divided into at least 3, at least 5, at least 10, at least 30, at least 50, at least 100, at least 300, at least 500, at least 1,000, at least 2,000, at least 3,000, at least 5,000, or at least 10,000 or more groups, and some of the groups may be exposed to different primers, e.g., to determine if different target nucleic acids are present or not.

Droplets containing both primer and a nucleic acid may then be treated to cause amplification of the nucleic acid to occur, e.g., if the primer is one that can recognize the nucleic acid within the droplet and allow amplification to occur. In some embodiments, even relatively rare nucleic acids (e.g., having mutations) may be determined, for example, from a sample containing larger numbers of non-mutated nucleic acids. Techniques for amplifying nucleic acids include PCR (polymerase chain reaction) or any of the other techniques described herein.

After amplification, the amplified nucleic acids may optionally be determined and/or sequenced, e.g., using techniques such as those described herein. In some embodiments, the droplets may be burst and the nucleic acids may be combined to facilitate determination and/or sequencing, although in some cases, the determination and/or sequencing may occur within the droplets.

Examples of methods for determining and/or sequencing nucleic acids include, but are not limited to, chain-termination sequencing, sequencing-by-hybridization, Maxam-Gilbert sequencing, dye-terminator sequencing, chain-termination methods, Massively Parallel Signature Sequencing (Lynx Therapeutics), polony sequencing, pyrosequencing, sequencing by ligation, ion semiconductor sequencing, DNA nanoball sequencing, single-molecule real-time sequencing (e.g., Pacbio sequencing), nanopore sequencing, Sanger sequencing, digital RNA sequencing (“digital RNA-seq”), Illumina sequencing, etc. In some cases, a microarray, such as a DNA microarray, may be used, for example, to determine, or to sequence, a nucleic acid.

Additional details regarding systems and methods for manipulating droplets in a microfluidic system follow, in accordance with certain aspects. For example, various systems and methods for screening and/or sorting droplets are described in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007, incorporated herein by reference. As a non-limiting example, in some aspects, by applying (or removing) a first electric field (or a portion thereof), a droplet may be directed to a first region or channel; by applying (or removing) a second electric field to the device (or a portion thereof), the droplet may be directed to a second region or channel; by applying a third electric field to the device (or a portion thereof), the droplet may be directed to a third region or channel; etc., where the electric fields may differ in some way, for example, in intensity, direction, frequency, duration, etc.

As mentioned, certain embodiments comprise a droplet contained within a carrying fluid. For example, there may be a first phase forming droplets contained within a second phase, where the surface between the phases comprises one or more proteins. For example, the second phase may comprise oil or a hydrophobic fluid, while the first phase may comprise water or another hydrophilic fluid (or vice versa). It should be understood that a hydrophilic fluid is a fluid that is substantially miscible in water and does not show phase separation with water at equilibrium under ambient conditions (typically 25° C. and 1 atm). Examples of hydrophilic fluids include, but are not limited to, water and other aqueous solutions comprising water, such as cell or biological media, ethanol, salt solutions, saline, blood, etc. In some cases, the fluid is biocompatible.

Similarly, a hydrophobic fluid is one that is substantially immiscible in water and will show phase separation with water at equilibrium under ambient conditions. As previously discussed, the hydrophobic fluid is sometimes referred to by those of ordinary skill in the art as the “oil phase” or simply as an oil. Non-limiting examples of hydrophobic fluids include oils such as hydrocarbons oils, silicon oils, fluorocarbon oils, organic solvents, perfluorinated oils, perfluorocarbons such as perfluoropolyether, etc. Additional examples of potentially suitable hydrocarbons include, but are not limited to, light mineral oil (Sigma), kerosene (Fluka), hexadecane (Sigma), decane (Sigma), undecane (Sigma), dodecane (Sigma), octane (Sigma), cyclohexane (Sigma), hexane (Sigma), or the like. Non-limiting examples of potentially suitable silicone oils include 2 cst polydimethylsiloxane oil (Sigma). Non-limiting examples of fluorocarbon oils include FC3283 (3M), FC40 (3M), Krytox GPL (Dupont), etc. In addition, other hydrophobic entities may be contained within the hydrophobic fluid in some embodiments. Non-limiting examples of other hydrophobic entities include drugs, immunologic adjuvants, or the like.

Thus, the hydrophobic fluid may be present as a separate phase from the hydrophilic fluid. In some embodiments, the hydrophobic fluid may be present as a separate layer, although in other embodiments, the hydrophobic fluid may be present as individual fluidic droplets contained within a continuous hydrophilic fluid, e.g. suspended or dispersed within the hydrophilic fluid. This is often referred to as an oil/water emulsion. The droplets may be relatively monodisperse, or be present in a variety of different sizes, volumes, or average diameters. In some cases, the droplets may have an overall average diameter of less than about 1 mm, or other dimensions as discussed herein. In some cases, a surfactant may be used to stabilize the hydrophobic droplets within the hydrophilic liquid, for example, to prevent spontaneous coalescence of the droplets. Non-limiting examples of surfactants include those discussed in U.S. Pat. Apl. Pub. No. 2010/0105112, incorporated herein by reference. Other non-limiting examples of surfactants include Span80 (Sigma), Span80/Tween-20 (Sigma), Span80/Triton X-100 (Sigma), Abil EM90 (Degussa), Abil we09 (Degussa), polyglycerol polyricinoleate “PGPR90” (Danisco), Tween-85, 749 Fluid (Dow Corning), the ammonium carboxylate salt of Krytox 157 FSL (Dupont), the ammonium carboxylate salt of Krytox 157 FSM (Dupont), or the ammonium carboxylate salt of Krytox 157 FSH (Dupont). In addition, the surfactant may be, for example, a peptide surfactant, bovine serum albumin (BSA), or human serum albumin.

The droplets may have any suitable shape and/or size. In some cases, the droplets may be microfluidic, and/or have an average diameter of less than about 1 mm. For instance, the droplet may have an average diameter of less than about 1 mm, less than about 700 micrometers, less than about 500 micrometers, less than about 300 micrometers, less than about 100 micrometers, less than about 70 micrometers, less than about 50 micrometers, less than about 30 micrometers, less than about 10 micrometers, less than about 5 micrometers, less than about 3 micrometers, less than about 1 micrometer, etc. The average diameter may also be greater than about 1 micrometer, greater than about 3 micrometers, greater than about 5 micrometers, greater than about 7 micrometers, greater than about 10 micrometers, greater than about 30 micrometers, greater than about 50 micrometers, greater than about 70 micrometers, greater than about 100 micrometers, greater than about 300 micrometers, greater than about 500 micrometers, greater than about 700 micrometers, or greater than about 1 mm in some cases. Combinations of any of these are also possible; for example, the diameter of the droplet may be between about 1 mm and about 100 micrometers. The diameter of a droplet, in a non-spherical droplet, may be taken as the diameter of a perfect mathematical sphere having the same volume as the non-spherical droplet.

In some embodiments, the droplets may be of substantially the same shape and/or size (i.e., “monodisperse”), or of different shapes and/or sizes, depending on the particular application. In some cases, the droplets may have a homogenous distribution of cross-sectional diameters, i.e., in some embodiments, the droplets may have a distribution of average diameters such that no more than about 20%, no more than about 10%, or no more than about 5% of the droplets may have an average diameter greater than about 120% or less than about 80%, greater than about 115% or less than about 85%, greater than about 110% or less than about 90%, greater than about 105% or less than about 95%, greater than about 103% or less than about 97%, or greater than about 101% or less than about 99% of the average diameter of the microfluidic droplets. Some techniques for producing homogenous distributions of cross-sectional diameters of droplets are disclosed in International Patent Application No. PCT/US2004/010903, filed Apr. 9, 2004, entitled “Formation and Control of Fluidic Species,” by Link, et al., published as WO 2004/091763 on Oct. 28, 2004, incorporated herein by reference. In addition, in some instances, the coefficient of variation of the average diameter of the droplets may be less than or equal to about 20%, less than or equal to about 15%, less than or equal to about 10%, less than or equal to about 5%, less than or equal to about 3%, or less than or equal to about 1%. However, in other embodiments, the droplets may not necessarily be substantially monodisperse, and may instead exhibit a range of different diameters.

Those of ordinary skill in the art will be able to determine the average diameter of a population of droplets, for example, using laser light scattering or other known techniques. The droplets so formed can be spherical, or non-spherical in certain cases. The diameter of a droplet, in a non-spherical droplet, may be taken as the diameter of a perfect mathematical sphere having the same volume as the non-spherical droplet.

In some embodiments, one or more droplets may be created within a channel by creating an electric charge on a fluid surrounded by a liquid, which may cause the fluid to separate into individual droplets within the liquid. In some embodiments, an electric field may be applied to the fluid to cause droplet formation to occur. The fluid can be present as a series of individual charged and/or electrically inducible droplets within the liquid. Electric charge may be created in the fluid within the liquid using any suitable technique, for example, by placing the fluid within an electric field (which may be AC, DC, etc.), and/or causing a reaction to occur that causes the fluid to have an electric charge.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, sawtooth, square, etc.), a DC field (i.e., one that is constant with respect to time), a pulsed field, etc. Techniques for producing a suitable electric field (which may be AC, DC, etc.) are known to those of ordinary skill in the art. For example, in one embodiment, an electric field is produced by applying voltage across a pair of electrodes, which may be positioned proximate a channel such that at least a portion of the electric field interacts with the channel. The electrodes can be fashioned from any suitable electrode material or materials known to those of ordinary skill in the art, including, but not limited to, silver, gold, copper, carbon, platinum, copper, tungsten, tin, cadmium, nickel, indium tin oxide (“ITO”), etc., as well as combinations thereof.

In another set of embodiments, droplets of fluid can be created from a fluid surrounded by a liquid within a channel by altering the channel dimensions in a manner that is able to induce the fluid to form individual droplets. The channel may, for example, be a channel that expands relative to the direction of flow, e.g., such that the fluid does not adhere to the channel walls and forms individual droplets instead, or a channel that narrows relative to the direction of flow, e.g., such that the fluid is forced to coalesce into individual droplets. In some cases, the channel dimensions may be altered with respect to time (for example, mechanically or electromechanically, pneumatically, etc.) in such a manner as to cause the formation of individual droplets to occur. For example, the channel may be mechanically contracted (“squeezed”) to cause droplet formation, or a fluid stream may be mechanically disrupted to cause droplet formation, for example, through the use of moving baffles, rotating blades, or the like.

Some embodiments generally relate to systems and methods for fusing or coalescing two or more droplets into one droplet, e.g., where the two or more droplets ordinarily are unable to fuse or coalesce, for example, due to composition, surface tension, droplet size, the presence or absence of surfactants, etc. In certain cases, the surface tension of the droplets, relative to the size of the droplets, may also prevent fusion or coalescence of the droplets from occurring.

As a non-limiting example, two droplets can be given opposite electric charges (i.e., positive and negative charges, not necessarily of the same magnitude), which can increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc. The droplets, in some cases, may not be able to fuse even if a surfactant is applied to lower the surface tension of the droplets. However, if the droplets are electrically charged with opposite charges (which can be, but are not necessarily of, the same magnitude), the droplets may be able to fuse or coalesce. As another example, the droplets may not necessarily be given opposite electric charges (and, in some cases, may not be given any electric charge), and are fused through the use of dipoles induced in the droplets that causes the droplets to coalesce. Also, the two or more droplets allowed to coalesce are not necessarily required to meet “head-on.” Any angle of contact, so long as at least some fusion of the droplets initially occurs, is sufficient. See also, e.g., U.S. patent application Ser. No. 11/698,298, filed Jan. 24, 2007, entitled “Fluidic Droplet Coalescence,” by Ahn, et al., published as U.S. Patent Application Publication No. 2007/0195127 on Aug. 23, 2007, incorporated herein by reference in its entirety.

In one set of embodiments, a fluid may be injected into a droplet. The fluid may be microinjected into the droplet in some cases, e.g., using a microneedle or other such device. In other cases, the fluid may be injected directly into a droplet using a fluidic channel as the droplet comes into contact with the fluidic channel. Other techniques of fluid injection are disclosed in, e.g., International Patent Application No. PCT/US2010/040006, filed Jun. 25, 2010, entitled “Fluid Injection,” by Weitz, et al., published as WO 2010/151776 on Dec. 29, 2010; or International Patent Application No. PCT/US2009/006649, filed Dec. 18, 2009, entitled “Particle-Assisted Nucleic Acid Sequencing,” by Weitz, et al., published as WO 2010/080134 on Jul. 15, 2010, each incorporated herein by reference in its entirety.

The following documents are each incorporated herein by reference in its entirety for all purposes: Int. Pat. Apl. Pub. No. WO 2016/168584, entitled “Barcoding System for Gene Sequencing and Other Applications,” by Weitz et al.; Int. Pat. Apl. Pub. No. WO 2015/161223, entitled “Methods and Systems for Droplet Tagging and Amplification,” by Weitz, et al.; U.S. Pat. Apl. Ser. No. 61/980,541, entitled “Methods and Systems for Droplet Tagging and Amplification,” by Weitz, et al.; U.S. Pat. Apl. Ser. No. 61/981,123, entitled “Systems and Methods for Droplet Tagging,” by Bernstein, et al.; Int. Pat. Apl. Pub. No. WO 2004/091763, entitled “Formation and Control of Fluidic Species,” by Link et al.; Int. Pat. Apl. Pub. No. WO 2004/002627, entitled “Method and Apparatus for Fluid Dispersion,” by Stone et al.; Int. Pat. Apl. Pub. No. WO 2006/096571, entitled “Method and Apparatus for Forming Multiple Emulsions,” by Weitz et al.; Int. Pat. Apl. Pub. No. WO 2005/021151, entitled “Electronic Control of Fluidic Species,” by Link et al.; Int. Pat. Apl. Pub. No. WO 2011/056546, entitled “Droplet Creation Techniques,” by Weitz, et al.; Int. Pat. Apl. Pub. No. WO 2010/033200, entitled “Creation of Libraries of Droplets and Related Species,” by Weitz, et al.; U.S. Pat. Apl. Pub. No. 2012-0132288, entitled “Fluid Injection,” by Weitz, et al.; Int. Pat. Apl. Pub. No. WO 2008/109176, entitled “Assay And Other Reactions Involving Droplets,” by Agresti, et al.; and Int. Pat. Apl. Pub. No. WO 2010/151776, entitled “Fluid Injection,” by Weitz, et al.; and U.S. Pat. Apl. Ser. No. 62/072,944, entitled “Systems and Methods for Barcoding Nucleic Acids,” by Weitz, et al.

In addition, the following are incorporated herein by reference in their entireties: U.S. Pat. Apl. Ser. No. 61/981,123 filed Apr. 17, 2014; PCT Pat. Apl. Ser. No. PCT/US2015/026338, filed Apr. 17, 2015, entitled “Systems and Methods for Droplet Tagging”; U.S. Pat. Apl. Ser. No. 61/981,108 filed Apr. 17, 2014; U.S. Pat. Apl. Ser. No. 62/072,944, filed Oct. 30, 2014; PCT Pat. Apl. Ser. No. PCT/US2015/026443, filed on Apr. 17, 2015, entitled “Systems and Methods for Barcoding Nucleic Acids”; U.S. Pat. Apl. Ser. No. 62/106,981, entitled “Systems, Methods, and Kits for Amplifying or Cloning Within Droplets,” by Weitz, et al.; U.S. Pat. Apl. Pub. No. 2010-0136544, entitled “Assay and Other Reactions Involving Droplets,” by Agresti, et al.; U.S. Pat. Apl. Ser. No. 61/981,108, entitled “Methods and Systems for Droplet Tagging and Amplification,” by Weitz, et al.; Int. Pat. Apl. Pub. No. PCT/US2014/037962, filed May 14, 2014, entitled “Rapid Production of Droplets,” by Weitz, et al.; and U.S. Provisional Patent Application Ser. No. 62/133,140, filed Mar. 13, 2015, entitled “Determination of Cells Using Amplification,” by Weitz, et al. Also, U.S. Provisional Patent Application Ser. No. 62/961,097, filed Jan. 14, 2020, entitled “Devices and Methods for Determining Nucleic Acids Using Digital Droplet PCR and Related Techniques,” by Weitz, et al., is incorporated herein by reference in its entirety.

The following examples are intended to illustrate certain embodiments of the present disclosure, but do not exemplify the full scope of the invention.

### Example 1

This example illustrates double digital droplet PCR, in accordance with one embodiment. The basic concept of this example is to use digital droplet PCR as the first stage of a two-stage detection scheme, followed by a second stage of detection. The first stage has all the advantages of normal digital PCR, as well as some less appreciated advantages. The second stage of detection allows much larger multiplexing in the first stage by doing the identification of the specific amplified target. It also allows methods that can increase both the specificity and the selectivity of the amplified targets.

In the first stage, the sample is compartmentalized, either into droplets or other compartments, so there is only one target oligonucleotide per drop. However, a large multiplex of primers can be used. In this case, the concentration of the initial oligonucleotide can be increased, since, while it is still essential to have at most one target per drop, there are many different targets, and only one of any of these can be in a drop.

This high degree of multiplexing provides some of the advantages of digital PCR including lack of sensitivity to amplification rate, since competitive amplification can be eliminated, and the lack of cross-amplification, which can introduce noise into the results. It also can allow for the sensitive amplification of very rare targets. In some or all of the compartments or droplets where amplification occurs, there is only one amplified target with, e.g., millions of copies of it.

The result provides for large amplification of targets, independently, but does not provide information about what the target is. Thus, identification of the target is done in the second stage of detection. Because there are large numbers of amplified targets, the detection of the target is much easier.

The second stage of detection can take many different forms, depending on the nature of the detection and the degree of quantification required.

For example, for sensitive and/or quantitative detection, a second stage of digital PCR can be performed. For this, the sample can be recombined, mixing all the contents of all the compartments or droplets together. For extra sensitivity, only those compartments with amplified targets can be selected, although this is not essential or required. Since there are a large number of amplified oligonucleotides, the sample can be divided into different samples, each of which can be detected, for instance, using standard digital PCR methods, with up to 4 colors for multiplexing in each channel. In some cases, the specificity can be improved by using nested primers to eliminate possible errors in the first stage. The results can be qualitative or quantitative.

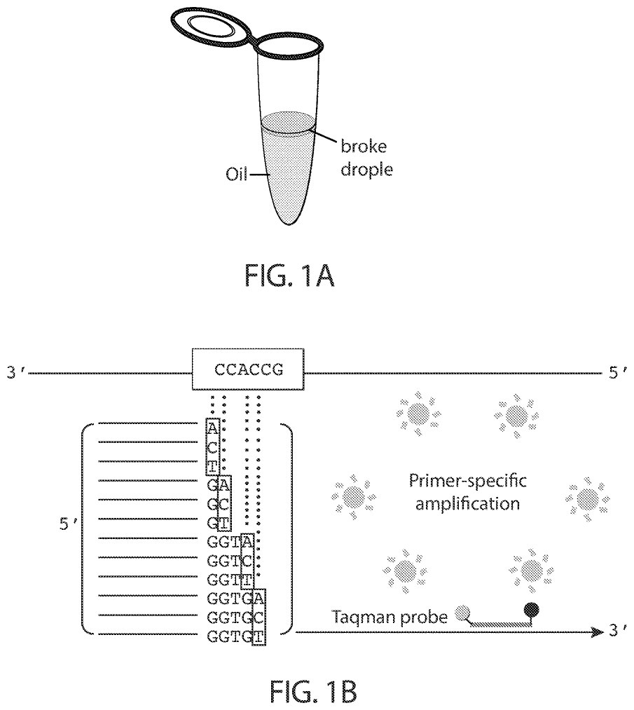

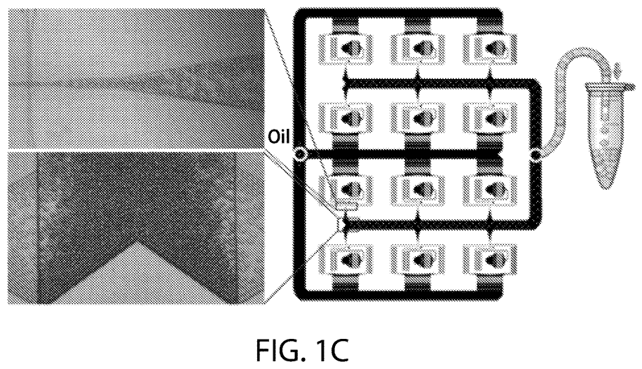

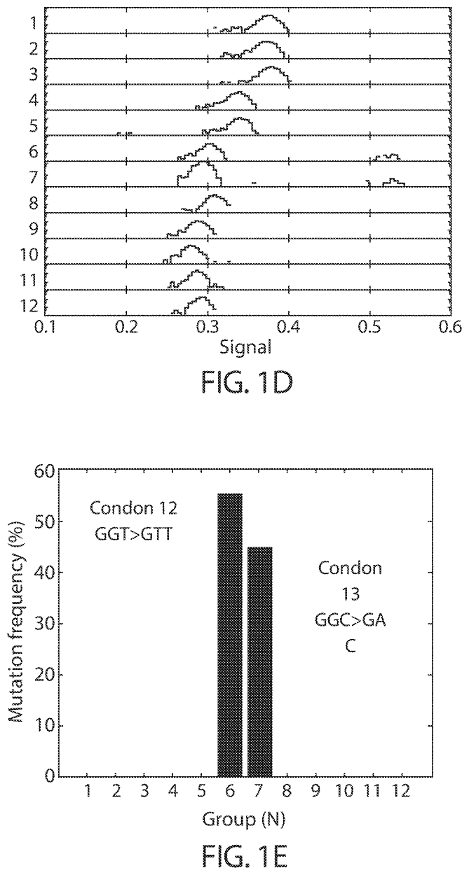

An example of this is summarized in FIG. 1, which shows how specific mutations in the KRAS gene can be detected using digital PCR. A first stage of digital PCR was performed using a blocking nucleotide that prevented amplification of the wide-type gene which has no mutations, but allowed all other mutations to be amplified. There were a total of 12 possible mutants that were studied. After this first digital PCR amplification stage, the contents of the compartments were combined (e.g., by breaking the droplets) to combine together all of the amplified mutants

A second stage of digital PCR was performed (FIG. 1C) with 12 independent drop makers, each using specific primers for one of the mutants. If this fashion, the mutants were individually identified (FIG. 1E).

It should be understood that this is by way of example, and other methods, such as the traditional Q-PCR or RT-PCR can also be used in other cases.

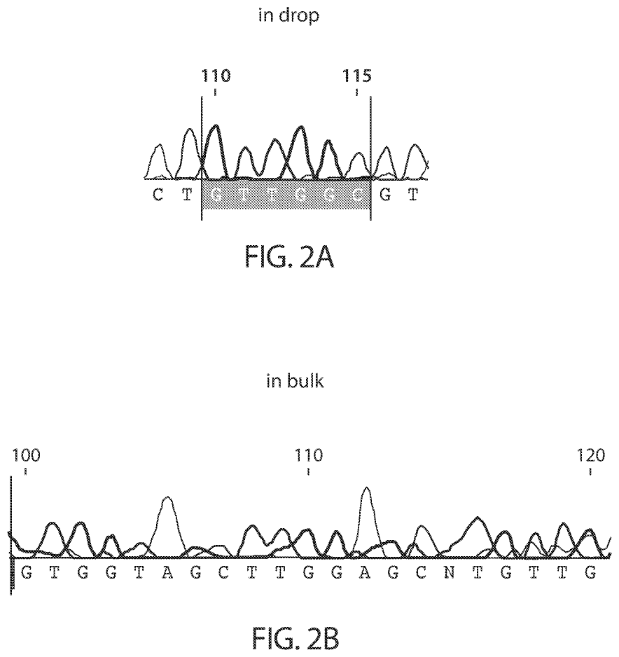

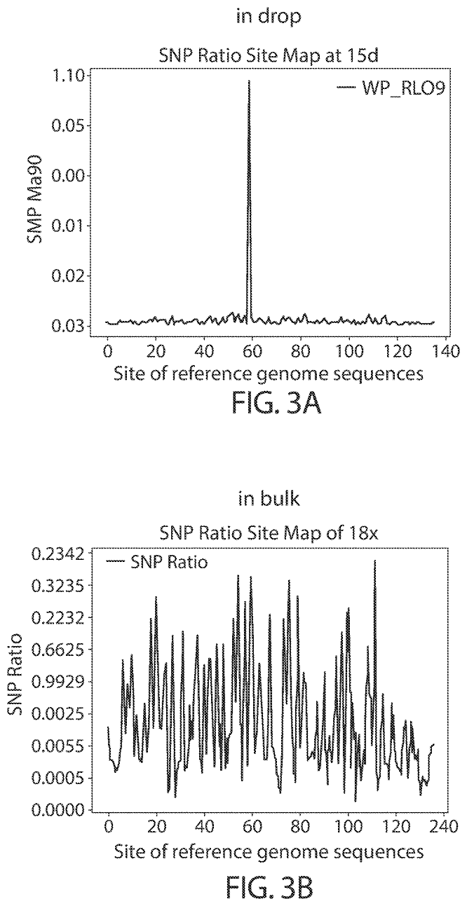

Various sequencing methods can also be used in other embodiments. For example, Sanger sequencing can be used to easily identify the amplified targets, as shown in FIG. 2. As another example, second generation or Illumina sequencing can be used. The initial isolation of the amplicons through targeted amplification may significantly increase the signal-to-noise ratio of the results, e.g., as shown in FIG. 3.

In some cases, all primer pairs can be added to each droplet. This results in much more sensitive detection while still increasing the signal-to-noise ratio.

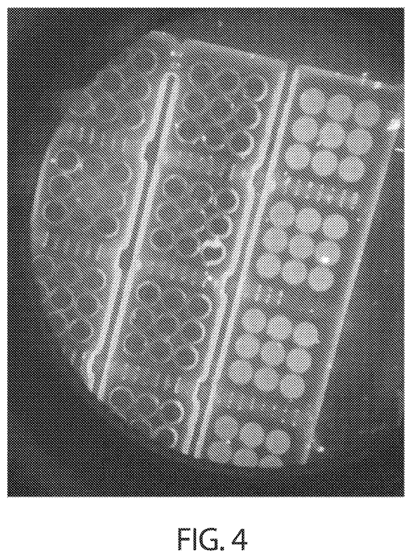

As another example, identification of the targets, without quantification, may be performed using a much simpler method, based on hybridization. Here, spatially separated regions of capture oligonucleotides may be arranged on a chip, such as a microarray chip. The droplets or compartments are merged and the solution, containing relatively large numbers of amplified targets, can be flowed over the chip. Specific targets are captured by hybridization oligonucleotides in known locations. Standard methods, such as fluorescence sandwich assays or enzymatic amplification assays, can be used to detect those regions that have captured targets, as shown in FIG. 4.

In summary, this example shows an initial stage of digital amplification through PCR in compartments or droplets using multiplexed primers, followed by a second stage of detection by merging the compartments or droplets.

### Example 2

In one set of experiments, a mixture of templates containing 0.1% mutant KRAS gene was encapsulated into droplets with a PCR mixture, followed by in-drop PCR and de-emulsification. Then, the collected aqueous phase was analyzed using Sanger sequencing. The in-droplet amplicons showed an expected result on the codon 12 (GTT), while the in-bulk amplicons show unrecognized peak. FIGS. 2A-2B show the Sanger sequencer results of the amplicon obtained from either in-drop amplification or in-bulk amplification. The mixture of templates contained 0.1% mutant KRAS gene.

This is shown in FIG. 1. FIG. 1 illustrates single-molecule characterization of each individual mutant amplicon using barcoded droplets. FIG. 1A shows that various possible mutant KRAS templates were amplified in droplets, then broken to collect the aqueous phase. FIG. 1B shows that primer-specific amplification is applied to characterize the various types of mutations in a single experiment. Each primer was designed to target one of twelve possible single-nucleotide mutations in codons 12 and 13. In FIG. 1C, to allow use of all twelve primers in a single amplification run, each primer was encapsulated with a different fluorescent barcode, producing twelve groups of barcoded droplets. In FIG. 1D, the histogram shows twelve distinct groups of droplets, and the droplets that show increased fluorescence signal in groups 6 and 7 revealed that there were two types of single-nucleotide mutations in this sample. FIG. 1E shows that the frequency of GGT-->GTT and GGT-->GAT is 55%, and 45%, respectively.

The target amplicon is between 110 and 115 as shown in FIG. 2A. The target is at a very low concentration in this sample, and can only be seen when the amplification is done in droplets. Then, each target molecule is isolated in a single drop and is amplified in that drop. In contrast, when amplification is done in bulk, the low concentration target molecules must compete with all other molecules and they are not well amplified, and hence are not visible in the Sanger sequencing shown in FIG. 2B.

Following are materials and methods using in this example and FIG. 1.

Preparation of DNA samples. Two human CRC cell lines HT29 and SW480 were purchased from ATCC, and cultivated in DMEM media supplemented with 10% fetal bovine serum. HT29 (ATCC HTB-38) has a wild-type KRAS gene, and SW480 (ATCC CCL-28) harbors a homozygous GTT mutant at codon 12 of KRAS gene. Genomic DNA (gDNA) was extracted from harvested cells using the QIAamp DNA Mini Kit (Qiagen) and eluted in AE buffer. The concentration of the gDNA was measured by a NanoDrop ND 1000 spectrophotometer.

Fabrication of microfluidic devices. Polydimethylsiloxane (PDMS) microfluidic devices were fabricated using standard soft lithographic methods. Briefly, SU8 photoresist (MicroChem) was spin-coated onto silicon wafer (University Wafer), patterned by OAI UV exposure through a photolithography mask, and developed. Then, Sylgard 184 silicone elastomer mixture (Dow Corning) at a weight ratio of Base:Curing agent=10:1 was poured onto the SU8 mold and degassed under vacuum. After curing for two hours at 65° C., the PDMS was gently peeled from the mold, and input/output ports were punched out of the PDMS with a 0.75 mm diameter Harris Uni-Core biopsy punch. The PDMS and glass sheet were plasma treated for 10 seconds, and then brought together for bonding. Finally, the microfluidic channel walls were made hydrophobic by treating them with PPG Aquapel.

Droplet-based peptide nucleic acid clamp PCR mixture. PCR primers and Taqman probes were synthesized by IDT, and the PNA was purchased from PNA Bio. The final volume of PNA clamp PCR mixture was 50 microliters containing 2 microliters of HotStarTaq Polymerase, 1×PCR buffer, 200 micromolar dNTPs, 0.4 micromolar forward and reverse primer, 1.2 micromolar PNA, 0.36 micromolar Taqman-MGB probe, 0.3 micrograms/microliter BSA, 1.5 microliters of 10% Tween 20, and 4.9 micrograms of gDNA.

Formation of monodisperse aqueous droplets and PCR. A self-assembly vacuum system was used to produce the droplets. The PCR mixture was loaded into a SCI 0.28×0.64 mm internal/external diameter PE/1 tubing (SCI), with one end inserted into the sample inlet of the droplet-making device. The fluorinated oil HFE-7500 containing 1% (w/w) surfactant was placed in a 10 mL plastic syringe with a BD 27G1/2 syringe needle and inserted into the oil inlet using a 0.38×1.09 mm internal/external diameter PE/2 tubing. A PCR tube was placed in another 10 mL plastic syringe which was equipped a T-branch pipe. A PE/2 tubing was glued on a 18 TW needle (Vita) and was inserted into the bottom of the PCR tube passing by the T-branch pipe. The other end of the PE/2 tubing was inserted into the device outlet. To suck the fluids through the drop maker to produce droplets, a wall-based vacuum was applied to the outlet. Then, the droplets generated in the microfluidic devices were collected in the PCR tube and afterwards covered by mineral oil, followed by thermocycling in a PCR machine. PCR was performed using an initial denaturation and enzyme activation step at 95° C. for 10 min, 40 cycles of 30 seconds at 95° C., 30 seconds at 55° C. for primer annealing, 50 seconds at 60° C. for elongation, and a final extension at 60° C. for 5 min.

Single-molecule characterization of each individual mutant amplicon using barcoded droplets. To break the sorted droplets, 20% of PFO was added, followed by vortex-mixing for 30 seconds and centrifugation for 5 min at 5,000 rpm. The phase-separated liquid was used as the template of PCR directly. Primer-specific amplification was applied to characterize all types of mutations in a single experiment. Each primer was designed to target one of twelve possible single-nucleotide mutations in codons 12 and 13. Successful amplification in droplets was detected using the same Taqman probe used in the first-round PNA-clamp PCR. To allow use of all twelve primers in a single amplification run, each primer was encapsulated with a different fluorescent barcode, producing twelve groups of barcoded droplets. The twelve barcodes used four concentrations of Texas red paired with three concentrations of Alexa 680, which were multiplexed together with the primers through a parallel droplet-making device. After thermocycling, droplet fluorescence was measured. Each of the twelve fluorescent barcodes indicated a different type of mutant nucleotide. The frequency of each mutant could thus be calculated by counting the number of bright green droplets within each barcoded group, and dividing by the total number of green droplets in all groups.

### Example 3

In this example, a mixture of template containing 0.1% mutant EGFR gene was encapsulated into droplets with a PCR mixture, followed by in-drop PCR and de-emulsification. Then, the collected aqueous phase was analyzed using next generation sequencing (NGS). The in-drop amplicons show an expected result shown by a unique peak, while the in-bulk amplicons show unrecognized peaks.

This can be seen in FIG. 3, which shows the NGS results of the amplicon obtained from either in-drop amplification or in-bulk amplification. A mixture of template containing 0.1% mutant EGFR gene were amplified with a mutation specific primer both in drop and in bulk, followed by NGS.

Following are materials and methods using in this example and FIG. 3.

Preparation of DNA samples. Two human CRC cell lines HT29 and SW480 were purchased from ATCC, and cultivated in DMEM media supplemented with 10% fetal bovine serum. HT29 (ATCC HTB-38) has a wild-type KRAS gene, and SW480 (ATCC CCL-28) harbors a homozygous GTT mutant at codon 12 of KRAS gene. Genomic DNA (gDNA) was extracted from harvested cells using the QIAamp DNA Mini Kit (Qiagen) and eluted in AE buffer. The concentration of the gDNA was measured by a NanoDrop ND 1000 spectrophotometer.

Fabrication of microfluidic devices. Polydimethylsiloxane (PDMS) microfluidic devices were fabricated using standard soft lithographic methods. Briefly, SU8 photoresist (MicroChem) was spin-coated onto silicon wafer (University Wafer), patterned by OAI UV exposure through a photolithography mask, and developed. Then Sylgard 184 silicone elastomer mixture (Dow Corning) at a weight ratio of Base:Curing agent=10:1 was poured onto SU8 mold and degassed under vacuum. After curing for two hours at 65° C., the PDMS was gently peeled from the mold and input/output ports were punched out of the PDMS with a 0.75 mm diameter Harris Uni-Core biopsy punch. The PDMS and glass sheet were plasma treated for 10 seconds, and then brought together for bonding. Finally, the microfluidic channel walls were made hydrophobic by treating them with PPG Aquapel.

Droplet-based peptide nucleic acid clamp PCR mixture. PCR primers and Taqman probes were synthesized by IDT, and the PNA was purchased from PNA Bio. The final volume of PNA clamp PCR mixture was 50 microliters containing 2 microliters of HotStarTaq Polymerase, 1×PCR buffer, 200 micromolar dNTPs, 0.4 micromolar forward and reverse primer, 1.2 micromolar PNA, 0.36 micromolar Taqman-MGB probe, 0.3 micrograms/microliter BSA, 1.5 microliters of 10% Tween 20, and 4.9 micrograms of gDNA.

Formation of monodisperse aqueous droplets and PCR. A self-assembly vacuum system was used to produce the droplets. The PCR mixture was loaded into a SCI 0.28×0.64 mm internal/external diameter PE/1 tubing (SCI), with one end inserted into the sample inlet of the droplet-making device. The fluorinated oil HFE-7500 containing 1% (w/w) surfactant is placed in a 10 mL plastic syringe with a BD 27G1/2 syringe needle and inserted into the oil inlet using a 0.38×1.09 mm internal/external diameter PE/2 tubing. A PCR tube was placed in another 10 mL plastic syringe which was equipped a T-branch pipe. A PE/2 tubing was glued on a 18 TW needle (Vita) and was inserted into the bottom of the PCR tube passing by the T-branch pipe. The other end of the PE/2 tubing was inserted into the device outlet. To suck the fluids through the drop maker to produce droplets, a wall-based vacuum was applied to the outlet. Then, the droplets generated in the microfluidic devices were collected in the PCR tube and afterwards covered by mineral oil, followed by thermocycling in a PCR machine. PCR was performed using an initial denaturation and enzyme activation step at 95° C. for 10 min, 40 cycles of 30 seconds at 95° C., 30 seconds at 55° C. for primer annealing, 50 seconds at 60° C. for elongation, and a final extension at 60° C. for 5 min.

Droplets breaking, PCR, and sequencing. To break the sorted droplets, 20% of PFO was added, followed by vortex-mixing for 30 seconds and centrifugation for 5 min at 5 000 rpm. The phase separated liquid was used as the template of PCR directly. If there was less than 5 microliters of liquid, 5 microliters of ddH₂O was added into it. The 50 microliters of PCR mixture included 2 microliters of Qiagen HotStarTaq Polymerase, 1×PCR buffer, 200 micromolar dNTPs, 0.4 micromolar forward and reverse primer, and 2 microliters of the liquid template. PCR was performed with preheating at 95° C. for 5 min, followed by 35 cycles of 95° C. for 40 seconds, 50° C. for 40 seconds, and 72° C. for 1 min, and a final extension at 72° C. for 7 min. Then, PCR amplicons were purified and sent to perform deep sequencing to confirm the status of codons 12 and 13.

### Example 4

In this example, HCV templates were encapsulated into droplets with a PCR mixture, followed by in-drop PCR and de-emulsification, using techniques similar to those described above. Then, the collected aqueous phase was introduced into a chip that comprised posts with a variety of types of probes. The posts that showed a positive signal carried specific probes for capturing the HCV amplicons, while other posts that showed a negative signal carried other different probes.

FIG. 4 shows hybridization results obtained by flowing the in-drop amplicons from a HCV plasmid onto a chip that composed of posts with different types of probes.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
forming a first plurality of droplets, at least 90% of which contain either only one target nucleic acid or no target nucleic acid, and at least 90% of which contain at least one amplification primer;
amplifying the target nucleic acids within the first plurality of droplets using the at least one amplification primer to produce amplified nucleic acids;
breaking the first plurality of droplets to mix the amplified nucleic acids;
forming a second plurality of droplets, at least 90% of which contains either one of the amplified nucleic acids or no amplified nucleic acid, and at least 90% of which contain at least one selection primer;
amplifying the amplified nucleic acids within the second plurality of droplets using the at least one selection primer to produce determinable nucleic acids; and
determining at least some of the determinable nucleic acids.

2. The method of claim 1, wherein forming the first plurality of droplets comprises merging one plurality of droplets, at least some of which contain either only one target nucleic acid or no target nucleic acid, with another plurality of droplets, at least some of which contain amplification primers, to form the first plurality of droplets.

3. The method of claim 1, wherein forming the first plurality of droplets comprises merging one plurality of droplets comprising, on average, less than one target nucleic acid, with another plurality of droplets, at least some of which contain amplification primers, to form the first plurality of droplets.

4. The method of claim 1, wherein forming the first plurality of droplets comprises merging one plurality of droplets comprising, on average, more than one target nucleic acid, with another plurality of droplets, at least some of which contain amplification primers, to form the first plurality of droplets.

5. The method of any one of claims 1-4, wherein in the another plurality of droplets, at least 90% of the droplets contains either one amplification primer or no amplification primer.

6. The method of any one of claims 1-5, wherein forming the first plurality of droplets comprises picoinjecting a fluid comprising amplification primers into a plurality of droplets, at least some of which contain either only one target nucleic acid or no target nucleic acid, to form the first plurality of droplets.

7. The method of any one of claims 1-6, wherein in the first plurality of droplets, at least 95% contains either only one target nucleic acid or no target nucleic acid.

8. The method of any one of claims 1-7, wherein in the first plurality of droplets, at least 50% contains only one target nucleic acid.

9. The method of any one of claims 1-8, wherein in the first plurality of droplets, at least 75% contains only one target nucleic acid.

10. The method of any one of claims 1-9, wherein in the first plurality of droplets, at least 95% contains only one target nucleic acid.

11. The method of any one of claims 1-10, wherein in the first plurality of droplets, at least 50% contains at least one amplification primer.

12. The method of any one of claims 1-11, wherein in the first plurality of droplets, at least 75% contains at least one amplification primer.

13. The method of any one of claims 1-12, wherein the first plurality of droplets comprises at least 3 amplification primers.

14. The method of any one of claims 1-13, wherein the first plurality of droplets comprises at least 5 amplification primers.

15. The method of any one of claims 1-14, wherein the first plurality of droplets comprises at least 10 amplification primers.

16. The method of any one of claims 1-15, wherein in the first plurality of droplets, at least some primers are attached to a nucleic acid barcode.

17. The method of claim 16, wherein in the first plurality of droplets, at least some primers are attached to a fluorescent tag.

18. The method of claim 17, wherein in the first plurality of droplets, amplification primers having different sequences are attached to distinguishable fluorescent tags.

19. The method of any one of claims 1-18, wherein in the first plurality of droplets, each of the amplification primers is identical to at least one other amplification primer within the first plurality of droplets except for a difference of only 1 or 2 nucleotides.

20. The method of any one of claims 1-19, wherein in the first plurality of droplets, each of the amplification primers is identical to at least one other amplification primer within the first plurality of droplets except for a difference of only 1 nucleotide.

21. The method of any one of claims 1-20, wherein amplifying the target nucleic acids within the droplets using the at least one amplification primer to produce amplified nucleic acids comprises amplifying the target nucleic acids using PCR.

22. The method of any one of claims 1-21, comprising breaking the first plurality of droplets using ultrasound.

23. The method of any one of claims 1-22, comprising breaking the first plurality of droplets by exposing the droplets to a surfactant.

24. The method of any one of claims 1-23, comprising breaking the first plurality of droplets using mechanical disruption.

25. The method of any one of claims 1-24, comprising forming the second plurality of droplets using flow focusing.

26. The method of any one of claims 1-25, comprising forming the second plurality of droplets using a plurality of flow focusing units.

27. The method of claim 26, wherein each of the plurality of flow focusing units incorporates a different selection primer into the second plurality of droplets

28. The method of any one of claims 25-27, comprising forming the second plurality of droplets using flow focusing, at least 90% of which contains either one of the amplified nucleic acids or no amplified nucleic acid.

29. The method of claim 28, further comprising merging a plurality of droplets with another plurality of droplets, at least some of which contain selection primers, to form the first plurality of droplets.

30. The method of claim 29, wherein in the another plurality of droplets, at least 90% of the droplets contains either one selection primer or no amplification primer.

31. The method of claim 30, comprising picoinjecting a fluid comprising selection primers into the another plurality of droplets.

32. The method of any one of claims 1-31, wherein in the second plurality of droplets, at least 95% contains either only one amplified nucleic acid or no amplified nucleic acid.

33. The method of any one of claims 1-32, wherein in the second plurality of droplets, at least 50% contains only one amplified nucleic acid.

34. The method of any one of claims 1-33, wherein in the second plurality of droplets, at least 75% contains only one amplified nucleic acid.

35. The method of any one of claims 1-34, wherein in the second plurality of droplets, at least 95% contains only one amplified nucleic acid.

36. The method of any one of claims 1-35, wherein in the second plurality of droplets, at least 50% contains at least one selection primer.

37. The method of any one of claims 1-36, wherein in the second plurality of droplets, at least 75% contains at least one selection primer.

38. The method of any one of claims 1-37, wherein amplifying the amplified nucleic acids within the droplets using the at least one selection primer to produce determinable nucleic acids comprises amplifying the amplified nucleic acids using PCR.

39. The method of any one of claims 1-38, wherein amplifying the amplified nucleic acids within the droplets using the at least one selection primer to produce determinable nucleic acids comprises amplifying the amplified nucleic acids using Q-PCR.

40. The method of any one of claims 1-39, wherein amplifying the amplified nucleic acids within the droplets using the at least one selection primer to produce determinable nucleic acids comprises amplifying the amplified nucleic acids using RT-PCR.

41. The method of any one of claims 1-40, comprising breaking the second plurality of droplets to mix the determinable nucleic acids.

42. The method of any one of claims 1-41, further comprising sequencing at least some of the determinable nucleic acids.

43. A method, comprising:
forming a plurality of droplets, at least 90% of which contain either only one target nucleic acid or no target nucleic acid, and at least 90% of which contain a plurality of different amplification primers;
amplifying the target nucleic acids within the plurality of droplets using the plurality of amplification primers to produce amplified nucleic acids;
breaking the droplets to form a mixture of the amplified nucleic acids; and
determining at least some of the amplified nucleic acids within the mixture.

44. The method of claim 43, wherein for droplets containing amplified nucleic acids, at least 90% of the amplified nucleic acids within a droplet are substantially identical.

45. The method of any one of claim 43 or 44, wherein at least 90% of the droplets each contain a plurality of different amplification primers able to recognize different target nucleic acids.

46. The method of any one of claims 43-45, wherein determining at least some of the amplified nucleic acids within the mixture comprises sequencing at least some of the amplified nucleic acids.

47. The method of claim 46, wherein determining at least some of the amplified nucleic acids within the mixture comprises sequencing at least some of the amplified nucleic acids using Sanger sequencing.

48. The method of any one of claim 46 or 47, comprising sequencing at least some of the amplified nucleic acids using Illumina sequencing.

49. The method of any one of claims 46-48, comprising sequencing at least some of the determinable nucleic acids using a DNA microarray.

50. The method of any one of claims 46-49, comprising sequencing at least some of the determinable nucleic acids using nanopore sequencing.

51. The method of any one of claims 46-50, comprising sequencing at least some of the determinable nucleic acids using capillary electrophoresis.

52. The method of any one of claims 46-51, comprising sequencing at least some of the determinable nucleic acids using single-molecule real-time sequencing.

53. The method of any one of claims 43-52, wherein determining at least some of the amplified nucleic acids comprises forming a second plurality of droplets encapsulating the mixture.

54. The method of claim 53, further comprising amplifying at least some of the encapsulated nucleic acids within the second plurality of droplets to produce determinable nucleic acids, and determining at least some of the determinable nucleic acids.

55. The method of any one of claim 53 or 54, comprising forming the second plurality of droplets such that at least 90% of the droplets contains either one of the amplified nucleic acids or no amplified nucleic acid.

56. The method of any one of claims 53-55, further comprising encapsulating at least one selection primer within the second plurality of droplets.

57. The method of claim 56, comprising dividing the second plurality of droplets into a plurality of groups of droplets, and exposing each group of droplets to a different selection primer.

58. The method of any one of claim 56 or 57, comprising dividing the second plurality of droplets into at least 5 groups.

59. The method of any one of claims 56-58, comprising dividing the second plurality of droplets into at least 10 groups.

60. The method of any one of claims 56-59, comprising dividing the second plurality of droplets into at least 30 groups.

61. The method of any one of claims 56-60, comprising dividing the second plurality of droplets into at least 100 groups.
